(19) United States
(12) Patent Application Publication
(10) Pub. No.: US 2024/0271293 A1
(43) Pub. Date: Aug. 15, 2024

(54) PRODUCTION OF AQUEOUS HYPOCHLOROUS ACID THROUGH THE ELECTROLYSIS OF PH MODIFIED BRINES

(71) Applicant: De Nora Water Technologies, LLC, Colmar, PA (US)

(72) Inventor: Andrew K. BOAL, Colmar, PA (US)

(21) Appl. No.: 18/562,915

(22) PCT Filed: May 20, 2022
(86) PCT No.: PCT/US2022/030176

§ 371 Date: Nov. 21, 2023

Related U.S. Application Data
(60) Provisional application No. 63/192,448, filed on May 24, 2021.

(51) Int. Cl. C25B 1/26; A61L 2/18; A61L 101/20; C25B 15/023

(52) U.S. Cl. CPC C25B 1/26; C25B 15/023; A61L 2/18; A61L 2101/20

(57) ABSTRACT

Methods and apparatuses that enable the production of aqueous halogen solutions with controlled pH values are disclosed. The disclosed methods include introducing acids into a halide-based brine, the electrolysis of which results in the production of an aqueous halogen solution product with a desired pH value, also preventing the pH of the product from reaching an undesired value. The disclosed technology can also utilize sensors and a control system utilizing telemetry from these sensors to ensure that the desired aqueous halogen solution is produced.

### RELATED APPLICATIONS

The present application claims priority to and the benefit of U.S. patent application No. 63/192,448, “Production Of Aqueous Hypochlorous Acid Through The Electrolysis Of pH Modified Brines” (filed May 24, 2021), the entirety of which application is incorporated herein by reference for any and all purposes.

### TECHNICAL FIELD

The present disclosure is related to the production of aqueous halogen solutions.

### BACKGROUND

Disinfection of aqueous fluids and solid surfaces is often accomplished by using aqueous halogen solutions, either by dosing halogen solutions into a larger body of aqueous fluid or through the direct application of an aqueous halogen solution to the surface to be disinfected. While any form of aqueous halogen can be used in this way, it is recognized by those in the fields that the hypohalous form of an aqueous halogen provides superior disinfection results compared to the hypohalite form of an aqueous halogen.

It is also recognized by those in the field that high concentrations of the molecular form of aqueous halogens (in particular chlorine) are disadvantageous in that molecular halogens have a relatively low solubility in water compared to the hypohalous and hypohalite forms and can off-gas from an aqueous solution, representing a potentially hazardous scenarios for anyone exposed to the gas. Further, it is recognized by those familiar with the art that electrohalogenation processes, which are understood in the context of the present disclosure to be a process whereby an electric current is applied to an aqueous halide solution to produce an aqueous halogen solution, are advantageous in the disinfection of aqueous fluids and surfaces because these processes enable on-demand production of aqueous halogen solutions.

It is also known in the art that electrohalogenation processes when run under normal operational conditions employing circum-neutral pH (6 to 8) halide solutions, will produce an aqueous halogen solution with an elevated pH, commonly in the range of 9 to 10. Aqueous halogen solutions in this pH range are, however, comprised almost completely of the hypohalite form of the halogen.

It will be recognized by those in the field that the production of an aqueous halogen solution of a lower pH value such that the pH is in a range wherein the aqueous halogen is present primarily in the form of hypohalous acid but with a pH that is not low enough such that significant amounts of molecular halogen (less than 1%) are present would be advantageous both in terms of applying the solution to aqueous fluids or surfaces for the purposes of achieving the disinfection of said aqueous fluids or surfaces. Accordingly, there is a long-felt need in the art for systems and methods for accomplishing such production.

### SUMMARY

In meeting the described long-felt needs, the present disclosure is directed to, inter alia, devices and methods by which it is possible to produce aqueous halogen solutions having a desired pH and a desired halogen concentration that would optimize those said solutions for the purposes of disinfecting either aqueous fluids or surfaces. Accordingly, the present disclosure provides the production of aqueous halogen solutions having a pH and halogen concentration in a range that is desirable for the disinfection of surfaces wherein the aqueous halogen solution is produced by modification of a halide containing brine prior to electrolysis through the introduction of acids (e.g., inorganic acids). As described, the disclosed technology can utilize sensors and controls to achieve the production of aqueous halogen solutions with specific pH values and halogen concentrations.

In one aspect, the present disclosure provides methods of forming a halogen solution, comprising: electrolyzing an aqueous feed comprising a halide: the electrolyzing being performed so as to give rise to a product primarily comprising a hypohalous acid; and modulating a pH of the feed and/or an operating condition of the electrolyzing such that the hypohalous acid concentration of the product is maintained in the range of from about 1,000 to about 10,000 mg/L, optionally in the range of from about 3,000 mg/L to about 8,000 mg/L.

Also provided are systems for the production of a halogen solution, comprising: an electrolysis cell, the electrolysis cell being configured to electrolyze a feed comprising a halide brine so as to give rise to a product having a hypohalous acid concentration: a source of the halide brine, the source of the halide brine in fluid communication with the electrolysis cell; and a sensor train configured to determine any one or more of feed pH, product pH, halogen concentration of the product, a current of the electrolysis cell, a voltage of the electrolysis cell, the system being configured to modulate a pH of the feed and/or a condition of the electrolysis cell such that the hypohalous acid concentration of the product is maintained in the range of from about 1,000 to about 10,000 mg/L.

Further provided are methods, comprising operating a system according to the present disclosure (e.g., any one of Aspects 23-42) so as to give rise to a product having a hypohalous acid concentration in the range of from about 1,000 to about 10,000 mg/L.

Also provided is a method of forming a halogen solution, comprising: electrolyzing an aqueous feed comprising a halogen in halide form so as to give rise to a product comprising the halogen, the halogen in the product being primarily in hypohalous acid form, as measured on a molar basis exclusive of water in the product, and the electrolyzing optionally being performed in an undivided cell; and modulating a pH of the feed and/or an operating condition of the electrolyzing such that the hypohalous acid concentration of the product is maintained in the range of from about 1,000 to about 10,000 mg/L, optionally in the range of from about 3,000 mg/L to about 8,000 mg/L.

Additionally disclosed is a system for the production of a halogen solution, comprising: an electrolysis cell, the electrolysis cell being configured to electrolyze an aqueous feed comprising a halogen in halide form so as to give rise to a product having a hypohalous acid concentration, and the electrolysis cell optionally being an undivided cell: a source of the halogen in halide form, the source of the halogen in halide form being capable of fluid communication with the electrolysis cell; and a sensor train configured to determine any one or more of feed pH, product pH, halogen content of the product, a current of the electrolysis cell, a voltage of the electrolysis cell, and the system being configured to modulate a pH of the feed and/or a condition of the electrolysis cell such that the hypohalous acid concentration of the product is maintained in the range of from about 1,000 to about 10,000 mg/L.

Also provided is a method, comprising operating a system according to the present disclosure (e.g., according to any one of Aspects 56-62) so as to give rise to a product having a hypohalous acid concentration in the range of from about 1,000 to about 10,000 mg/L, optionally in the range of from about 3,000 mg/L to about 8,000 mg/L.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating a preferred embodiment of the disclosure and are not to be construed as limiting the invention. In the drawings:

FIG. 1 is a schematic drawing of a system for the production of pH-controlled aqueous halogen solutions using a single chemical input to the system feed water.

FIG. 2 is a schematic drawing of a system for the production of pH-controlled aqueous halogen solutions using a dual chemical input to the system feed water.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated+10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value: they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Embodiments of the disclosed technology, as well as the practice of the disclosed technology, are intended to produce aqueous halogen solutions having a desired pH and halogen content through the use of an electrohalognenation process wherein aqueous halide solutions are electrolyzed to produce the desired solutions. Within the scope of the disclosed technology, the desired pH and halogen content of the solutions produced through the practice of the disclosed technology will be understood as solutions that are primarily comprised (>90%) of hypohalous acid forms of the aqueous halogen while minimizing the content of both hypohalite ions and molecular halogens in the solution with the solution also having a hypohalous acid concentration in the range of between 1,000 and 10,000 mg/L. Wherein the aqueous halogen solutions produced through the practice of the disclosed technology are aqueous chlorine solution, the desirable pH range is between 5 and 7.

Individuals skilled in the art will recognize that an aqueous chlorine solution having a chlorine concentration in the range of 1,000 and 10,000 mg/L and a pH in the range of 5 and 7 is the preferred composition of aqueous chlorine solutions for a variety of applications and especially for surface disinfection. As an example, the United States Centers for Disease Control (CDC) recommends that an aqueous chlorine solution with a chlorine concentration of 0.5% or 5,000 mg/L be used for surface disinfection applications.

With regards to pH, those skilled in the art also recognize that hypochlorous acid is generally a much more effective disinfection agent compared to the hypochlorite ion. Hypochlorous acid has a pKa of about 7.5, meaning that when the pH of the aqueous chlorine solution is below 7.5, the predominant form of chlorine in that solution is hypochlorous acid. Similarly, when the pH of the aqueous chlorine solution is around 7.0, hypochlorous acid makes up approximately 90% of the chlorine present in solution, with the remaining 10% being the hypochlorite ion. Therefore, an aqueous solution with a pH of less than 7 would be preferable in order to ensure that the predominate chlorine species present is also the more biocidally active species. Those skilled in the art will also realize that, when the pH of an aqueous chlorine solution falls below about 4, appreciable amounts of molecular chlorine can be present in the solution. Molecular chlorine has a low solubility in water, which may cause the evolution of chlorine gas and result in a hazard. Therefore, it will be recognized by those skilled in the art that the preferable pH of an aqueous chlorine solution not approach 4 and will preferably be higher such as 5 where the predominant chlorine species is still hypochlorous acid but there is no risk in evolving chlorine gas.

Production of aqueous halogen solutions through electrohalogenation processes is known in the art to proceed through a combination of halide ion oxidation on anodes and water reduction on cathodes, with the anodic process producing molecular halogens and the cathodic process producing hydroxide ions. For example, in electrohalogenation processes based on the oxidation of chloride ions, the anodic electrochemical reaction for chloride is:

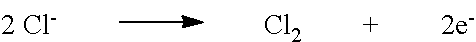

On the cathode side, the reduction of water occurs according to this reaction:

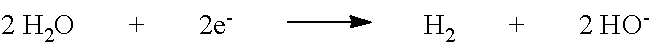

When electrohalogenation processes are practiced using cells that are chemically but not electrically isolated, such as in the case of a membrane divided cell, the products of the anodic and cathodic processes are collected separately as solutions of low pH anolyte, containing the halogen product, and high pH catholyte, which contains the hydroxide product. In an undivided electrolytic cell, such as disclosed herein, the products of the anolyte and catholyte processes combine to produce hypohalite ions. Practitioners familiar with the art understand that the product solution from an undivided electrolytic cell can be 2-3 pH units higher than the brine pre-electrolysis and that, in the case of chloride ion electrolysis processes, the pH of the product hypochlorite solution is typically in the range of 8 to 10, which is several pH units higher than the desirable range of 5-7 for the production of a product solution primarily comprised of hypochlorous acid.

The disclosed technology provides, inter alia, the use of acids combined with pH sensors to control the pH of the product solution so that the pH of the solution produced through electrolysis will be in a pH range where the primary aqueous halogen component is the hypohalous form of the halogen and also limits the pH from dropping too low so that the presence of molecular halogen in the aqueous halogen solution is not problematic when practicing the invention. It is a further objective of the disclosed technology to provide the production of aqueous halogen solution with the desired pH at a halogen concentration of between 1,000 and 10,000 mg/L, which is a concentration suitable for the disinfection of aqueous fluids and surfaces. It is a further objective of the disclosed technology to provide the use of inorganic acids to achieve the production of the desired halogen solutions so as to minimize the production of undesirable halogenated organic compounds, such as haloacetic acids, which can be produced as a result of using organic acids (e.g., acetic acid) in the production of the desired halogen solutions.

The disclosed technology can operate without the use of organic acids to adjust the pH of both the brine and resulting aqueous halogen solution. Organic acids (acetic acid, in particular) in the presence of aqueous halogen solutions can result in the undesirable production of a class of chemicals called haloacetic acids, which are strictly regulated due to their known carcinogenic properties. One skilled in the art will recognize that not forming haloacetic acids in a hypohalous acid solution, especially if the use of the solution is to spray it on a surface where it might be possible for the user to inhale the mist, would be an improvement over existing approaches

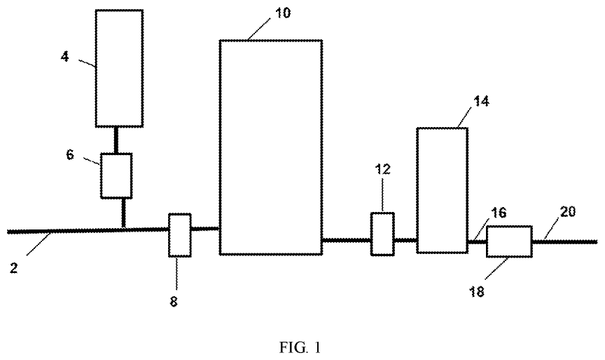

In the non-limiting embodiment of the disclosed technology shown in FIG. 1, line 2 is used to deliver fresh water. Tank 4 is a reservoir of a halide ion containing solution, preferably formulated with sodium chloride as the primary halide containing component of the brine compounded with a strong or weak acid. In the practice of the disclosed technology, the strong or weak acid can be selected from a variety of acids including, but not limited to, hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or combinations thereof.

Strong acids (e.g., strong mineral acids) and weak buffering acids can be used together, e.g., hydrochloric acid and dihydrogen potassium phosphate, as shown in Example 7 herein. A strong acid (e.g., an inorganic acid) and a related base/salt (e.g., phosphoric acid and sodium phosphate: sulfuric acid and one or more sulfate salts) can be used together, although this is not a requirement.

Pump 6 is used to inject the halide ion containing solution into the feed water in line 2, and the combined flow then passes through (optional) sensor package 8, which contains a variety of sensors to monitor physical and chemical aspects of the aqueous solution including, but not limited to pH, conductivity, temperature, and flow rate. The solution then passes through electrolytic cell 10, where a current is applied to solution causing the oxidation of chloride ions contained in the solution. The solution then exits electrolytic cell 10 where the solution passes a second sensor package 12 (also optional) before being collected in tank 14. Sensor package 12 contains a variety of sensors to monitor physical and chemical aspects of the aqueous solution including, but not limited to pH, conductivity, temperature, and flow rate. Telemetry from sensor packages 8 and 12 can be used to control the operation of the invention to ensure that solution with the desired chemical composition is produced. The product halogen solution can have a pH in the range of 5 and 7 with a halogen content of between 1,000 and 10,000 mg/L in the case of chlorine and a pH in the range of 6 and 8 with a halogen content of between 1,000 and 10,000 mg/L in the case of bromine. Line 16 can be used to communicate material from tank 14 to pump 18, which in turn encourages material to a use location via line 20.

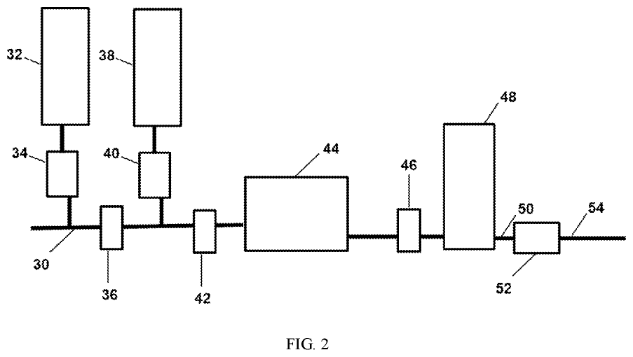

An alternative embodiment of the disclosed technology is shown in FIG. 2. Here, line 30 is used to deliver freshwater to the invention. Tank 32 is a reservoir containing an aqueous acid solution formulated from a variety of strong and weak acids selected from, but not limited to, hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or combinations thereof. This solution is injected into the feed water in line 30 through the action of pump 34. After the addition of aqueous acid to the water, the solution passes through sensor package 8, which contains a variety of sensors to monitor physical and chemical aspects of the aqueous solution including, but not limited to pH, conductivity, temperature, and flow rate. Tank 38 contains a halide ion brine solution preferably formulated from sodium chloride, although the solution can be formulated using any halide ion containing salt. This solution is injected into line 30 through the action of pump 40, and afterward passes through sensor package 42, which contains a variety of sensors to monitor physical and chemical aspects of the aqueous solution including, but not limited to pH, conductivity, temperature, and flow rate. The solution then passes through electrolytic cell 44, where a current is applied to solution causing the oxidation of chloride ions contained in the solution. The solution then exits electrolytic cell 44 where it passes a second sensor package 46 before being collected in tank 48. Sensor package 48 can include one or more sensors to monitor physical and chemical aspects of the aqueous solution, including (but not limited to) pH, conductivity, temperature, and flow rate. Telemetry from sensor packages 36, 42, and 46 can be used to control the operation of the system to ensure that solution with the desired chemical composition is produced, e.g., by varying the injection rates of acid from tank 32 and halide ion brine from tank 38. In some embodiments, the desired aqueous halogen solution will have a pH in the range of 5 and 7 with a halogen content of between 1,000 and 10,000 mg/L in the case of chlorine and a pH in the range of 6 and 8 with a halogen content of between 1,000 and 10,000 mg/L in the case of bromine. The telemetry can also be used to modulate the operation of electrolytic cell 44. Line 50 can be used to communicate material from tank 48 to pump 52, which in turn encourages material to a use location via line 54.

A system can operate based on monitoring only pH and chlorine (FAC) content, although this is not a requirement. Flow rates, temperature and conductivity can also be used in the overall control logic for an electrolysis system, but are not required for the disclosed technology. In the case of conductivity, conductivity can be measured indirectly by the system in terms of the current passed by the cell, and this is the primary control process for an electrolysis system. A call can operate at a fixed voltage and then measures the current that is passed in the cell with a goal of achieving a specific target number. If the current passed is too low; the system can increase the feed of chloride brine: if the current is too high, chloride brine injection can be decreased. Similarly, temperature and flow rate can be monitored (though this is not necessary) in an electrolysis system to ensure that no operational faults are occurring or, in the case of temperature, to ensure that the system does not operate outside of specification parameters.

A system can be used to formulate product that is stored and then distributed and/or dispensed to a use location. For example, a system can include a storage tank that stores product. The stored product can then be distributed (via piping, via portable containers) and then applied to use locations. A system according to the present disclosure can also be portable, e.g., carried by a person and/or mounted on wheels or rollers. Thus, the disclosed technology allows for formation of product in situ, but can also be used to form product that is stored for later use. A system can be automated, e.g., a system that dispenses product to certain locations (e.g., food preparation areas) on a set schedule. A system can also be manually operated, e.g., a system that a user controls for product production when needed.

### Example 1

An aqueous chlorine solution was prepared using an on-site generation system similar to the one described in FIG. 1. Electrolysis was carried out using an unmodified feed water source and a brine solution that was comprised of saturated sodium chloride brine and sodium bisulfate at a concentration of up to 75 g/L with an applied cell voltage of 13-15 V (plate to plate voltage of 4.7-5.0 V); in all cases, the cell current ranged from 9.9 to 10.2 A. Duplicate samples of aqueous chlorine solutions produced using these inputs were collected and characterized by measuring the pH and FAC content of the solutions (Table 1). As can be seen in the data, while the added sodium bisulfate was able to decrease the pH of the aqueous chlorine solution, the decrease was not linear with the amount of acid added to the sodium chloride brine.

TABLE 1

| Brine Sodium | Aqueous | Aqueous Chlorine |
|---|---|---|
| Bisulfate | Chlorine | Solution |
| Content (g/L) | Solution pH | FAC (mg/L) |
| 0 | 9.45 | 3,388 |
| 5 | 8.805 | 3,388 |
| 10 | 8.57 | 3,350 |
| 20 | 8.285 | 3,375 |
| 30 | 8.03 | 3,200 |
| 40 | 7.825 | 3,188 |
| 50 | 7.745 | 3,025 |
| 75 | 7.41 | 2,850 |

### Example 2

A series of aqueous chlorine solutions were prepared using an on-site generation system similar to the one described in FIG. 1. Electrolysis was carried out using an unmodified feed water source and a brine solution that was comprised of saturated sodium chloride brine and potassium dihydrogen phosphate at a concentration of up to 45.3 g/L with an applied cell voltage of 13-15 V (plate to plate voltage of 4.7-5.0 V) and in all cases, the cell current ranged from 9.9 to 10.1 A. Brine samples were characterized by measuring their pH values. Duplicate samples of aqueous chlorine solutions produced using these inputs were collected and characterized by measuring the pH and FAC content of the solutions (Table 2). Data from this example shows that potassium dihydrogen phosphate modified brines can be used to decrease the pH of aqueous chlorine solutions.

TABLE 2

| Brine Potassium | | Aqueous | Aqueous Chlorine |
|---|---|---|---|
| Dihydrogen Phosphate | Brine | Chlorine | Solution |
| Content (g/L) | pH | Solution pH | FAC (mg/L) |
| 0 | 7.42 | 9.47 | 3,775 |
| 11.3 | 3.69 | 8.62 | 3,800 |
| 45.3 | 3.22 | 7.96 | 3,763 |

### Example 3

A series of aqueous chlorine solutions were prepared using an on-site generation system similar to the one described in FIG. 1. Electrolysis was carried out using feed water that had been pH modified using either hydrochloric acid or sodium hydroxide to achieve a pH value in the range of 1.35 and 8.96 along with an unmodified saturated sodium chloride brine with an applied cell voltage 13-15 V (plate to plate voltage of 4.7-5.0 V) and in all cases, the cell current ranged from 10.0 to 10.5 A. Aqueous chlorine solutions produced using these inputs were collected and characterized by measuring the pH and FAC content of the solutions (Table 3). Data from this example shows that adjustment of the pH of the input feed water with strong acids or bases can be used to alter the pH of the aqueous chlorine solution to produce a solution with a pH in the desired range of 5 to 7 after electrolysis but that the relationship between feed water pH and aqueous chlorine solution is non-linear. Without being bound to any particular theory or embodiment, a comparatively small change in the pH of the feed water (i.e., from 1.53 to 1.35) resulted in a sizeable change in the pH of the product chlorine solution (i.e., from 6.94 to 4.04), demonstrating that using strong acids alone can, in some circumstances, lead to the production of chorine solutions with pH values below the desirable range if the use of the strong acid is not controlled.

TABLE 3

| Feed | Aqueous | Aqueous Chlorine |
|---|---|---|
| Water | Chlorine | Solution |
| pH | Solution pH | FAC (mg/L) |
| 1.35 | 4.04 | 3,625 |
| 1.53 | 6.94 | 3,737 |
| 2.01 | 7.64 | 3,213 |
| 3.07 | 8.63 | 3,100 |
| 4.02 | 8.85 | 3,168 |
| 4.94 | 8.92 | 3,300 |
| 6.02 | 9.06 | 3,275 |
| 7.03 | 9.24 | 3,688 |
| 8.05 | 9.35 | 3,588 |
| 8.96 | 9.38 | 3,638 |

Without being bound to any particular theory or embodiment, a change in the pH of the feed can result in an equal or greater change in the pH of the product halogen solution, although this has not been previously explored. Thus, brine/feed pH can have an influence on product oxidant pH. In this way, a user can select and/or modulate the pH of the feed (i.e., the pH of the brine, the pH of any water or acid added to the brine) so as to arrive at a product pH that is suitable for the user's needs. In some instances, the electrolytic cell (e.g., an undivided cell) can give rise to a product with a pH that is about 2 to 3 pH units higher than the pre-electrolysis brine. Thus, the pH of the feed and the pH of the product can differ by from, e.g., about 1 to about 5 pH units, about 1.3 to about 4.7 pH units, about 1.5 to about 4.5 pH units, about 1.8 to about 4.2 pH units, about 2 to about 4 pH units, about 2.2 to about 3.8 pH units, about 2.4 to about 3.6 pH units, about 2.6 to about 3.4 pH units, about 2.8 to about 3.2 pH units, or even about 3 pH units.

### Example 4

A series of aqueous chlorine solutions were prepared using an on-site generation system similar to the one described in FIG. 1. Electrolysis was carried out using feed water that had been pH modified using sodium bisulfate to achieve a pH value of between 1.62 and 7.07 along with an unmodified saturated sodium chloride brine with an applied cell voltage of 13-15 V (plate to plate voltage of 4.7-5.0 V) and in all cases, the cell current ranged from 10 to 10.1 A. Aqueous chlorine solutions produced using these inputs were collected and characterized by measuring the pH and FAC content of the solutions (Table 4). Data from this testing confirms that adjustment of the pH of the input feed water with a strong acid can be used to alter the pH of the aqueous chlorine solution resulting from electrolysis to produce a solution with a pH in the desired range of 5 to 7 and that the relationship between feed water pH and aqueous chlorine solution is non-linear. Moreover, this example also shows that it is possible to over acidify the inputs to the on-site generation system, resulting in a scenario where the pH of the aqueous chlorine solution is less than four and can result in the evolution of chlorine gas. Also as seen in Example 3, a small change in the pH of the feed water (i.e., from 1.74 to 1.62) resulted in a sizeable change in the pH of the product chlorine solution (i.e., from 6.43 to 2.01), demonstrating that using strong acids alone can lead to the production of chorine solutions with pH values below the desirable range if the use of the strong acid is not controlled.

TABLE 4

| Feed | Aqueous | Aqueous Chlorine |
|---|---|---|
| Water | Chlorine | Solution |
| pH | Solution pH | FAC (mg/L) |
| 1.62 | 2.01 | 678 |
| 1.74 | 6.43 | 3,075 |
| 1.96 | 7.37 | 3,475 |
| 3.07 | 8.59 | 3,788 |
| 4.00 | 8.83 | 3,713 |
| 5.02 | 8.86 | 3,675 |
| 6.07 | 8.96 | 3,788 |
| 7.07 | 9.20 | 3,738 |

### Example 5

A series of aqueous chlorine solutions were prepared using an on-site generation system similar to the one described in FIG. 1. Electrolysis was carried out using feed water that had been pH modified using potassium dihydrogen phosphate to achieve a pH value of between 1.62 and 7.07 along with an unmodified saturated sodium chloride brine with an applied cell voltage of 13-15 V (plate to plate voltage of 4.7-5.0 V) and in all cases, the cell current ranged from 10.0 to 10.1 A. Aqueous chlorine solutions produced using these inputs were collected and characterized by measuring the pH and FAC content of the solutions (Table 5). Data from this testing confirms that adjustment of the pH of the input feed water with a weak, buffering acid can be used to alter the pH of the aqueous chlorine solution to the desired range of between 5 and 7 after from electrolysis but, in the case of a weak acid, a significant amount of that acid may be required to achieve this desired outcome.

TABLE 5

| Feed | Aqueous | Aqueous Chlorine |
|---|---|---|
| Water | Chlorine | Solution |
| pH | Solution pH | FAC (mg/L) |
| 5.07 | 6.35 | 3,358 |
| 5.37 | 6.84 | 3,275 |
| 5.59 | 7.17 | 3,300 |
| 6.04 | 7.49 | 3,625 |
| 6.93 | 9.12 | 3,675 |

### Example 6

A series of aqueous chlorine solutions were prepared using an on-site generation system similar to the one described in FIG. 1. Electrolysis was carried out using feed water that had been pH modified using a combination of hydrochloric acid and potassium dihydrogen phosphate to achieve a pH value of between 3.05 and 5.99 along with an unmodified saturated sodium chloride brine with an applied cell voltage of 13-15 V (plate to plate voltage of 4.7-5.0 V) and in all cases, the cell current ranged from 9.9 to 10.2 A. Aqueous chlorine solutions produced using these inputs were collected and characterized by measuring the pH and FAC content of the solutions (Table 6). Data from this test demonstrates that an aqueous chlorine solution with pH and chlorine content in the desired range can be achieved by using a combination of a strong acid and a weak acid added together into the feed water for the electrolysis system. In this way, a halide brine (e.g., chloride brine) can be combined with a weak acid and/or a strong acid so as to achieve a feed with the desired pH, which feed can then be electrolyzed to give rise to the desired product solution.

TABLE 6

| Feed Water | Feed | Aqueous | Aqueous Chlorine |
|---|---|---|---|
| KH₂PO₄ | Water | Chlorine | Solution |
| Content (g/L) | pH | Solution pH | FAC (mg/L) |
| 1 | 3.05 | 8.04 | 3,563 |
| 1 | 5.99 | 8.19 | 3,500 |
| 10 | 3.05 | 6.61 | 3,350 |
| 10 | 5.97 | 6.91 | 3,275 |

### Example 7

A series of aqueous chlorine solutions were prepared using an on-site generation system similar to the one described in FIG. 1. Electrolysis was carried out using feed water that had been pH modified using hydrochloric acid to achieve a pH value of between 2.05 and 6.04 along with a saturated sodium chloride brine that had been modified by adding potassium dihydrogen phosphate at a concentration of either 10 g/L or 40 g/L with an applied cell voltage of 13-15 V (plate to plate voltage of 4.7-5.0 V) and in all cases, the cell current ranged from 9.9 to 10.2 A. Aqueous chlorine solutions produced using these inputs were collected and characterized by measuring the pH and FAC content of the solutions (Table 7). While aqueous chlorine solutions of the desired pH were not produced in this example, this example does demonstrate that the acids can be introduced into the brine to be electrolyzed through different injection points and have the desired outcome of lowering the pH of the product aqueous halogen solution.

TABLE 7

| Brine | Feed | Aqueous | Aqueous Chlorine |
|---|---|---|---|
| KH₂PO₄ | Water | Chlorine | Solution |
| Content (g/L) | pH | Solution pH | FAC (mg/L) |
| 10 | 2.04 | 7.80 | 3,488 |
| 10 | 4.02 | 8.42 | 3,425 |
| 10 | 5.98 | 8.49 | 3,363 |
| 40 | 2.03 | 7.52 | 3,588 |
| 40 | 4.06 | 7.89 | 3,588 |
| 40 | 6.04 | 7.91 | 3,575 |

### Aspects

The following Aspects are illustrative only and do not limit the scope of the present disclosure or the appended claims.

Aspect 1. A method of forming a halogen solution, comprising: electrolyzing an aqueous feed comprising a halide: the electrolyzing being performed so as to give rise to a product primarily comprising a hypohalous acid; and modulating a pH of the feed and/or an operating condition of the electrolyzing such that the hypohalous acid concentration of the product is maintained in the range of from about 1,000 to about 10,000 mg/L, optionally in the range of from about 3,000 mg/L to about 8,000 mg/L.

Aspect 2. The method of Aspect 1, wherein a halide brine and a feed water are contacted so as to form the feed, the feed water optionally comprising a feed acid.

Aspect 3. The method of Aspect 2, wherein the feed acid is an inorganic acid.

Aspect 4. The method of Aspect 2, wherein the feed acid comprises hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or combinations thereof.

Aspect 5. The method of Aspect 2, wherein the halide brine comprises a brine acid.

Aspect 6. The method of Aspect 5, wherein the brine acid is an inorganic acid.

Aspect 7. The method of Aspect 5, wherein the brine acid comprises hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or combinations thereof.

Aspect 8. The method of any one of Aspects 1-7, wherein the product defines a pH of from about 4 to about 8.

Aspect 9. The method of any one of Aspects 1-8, wherein the halide is chloride and wherein the product defines a pH of from about 5 to about 6.

Aspect 10. The method of any one of Aspects 1-8, wherein the halide is bromide and wherein the product defines a pH of from about 6 to about 7.

Aspect 11. The method of any one of Aspects 1-8, wherein the hypohalous acid comprises hypochlorous acid and wherein the product defines a pH of above about 4.

Aspect 12. The method of Aspect 11, wherein the product defines a pH of from about 5 to about 6.

Aspect 13. The method of any one of Aspects 1-8, wherein the hypohalous acid comprises hypobromous acid and wherein the product defines a pH of above about 4.

Aspect 14. The method of Aspect 13, wherein the product defines a pH of from about 6 to about 7.

Aspect 15. The method of any one of Aspects 1-14, wherein the product comprises an amount of halogen, and wherein the amount of halogen in the product is less than about 10 wt % (i.e., on a weight basis) molecular halogen, e.g., less than about 10 wt % molecular halogen, less than about 5% wt % molecular halogen, or less than about 1 wt % molecular halogen. Preferably, molecular halogen is less than about 5 wt % or less than about 1 wt % of the amount of halogen in the product.

Aspect 16. The method of any one of Aspects 1-15, wherein the product comprises an amount of halogen, and wherein the amount of halogen in the product is less than about 10 wt % (i.e., on a weight basis) hypohalite ion, e.g., less than about 10 wt % hypohalite ion, less than about 5 wt % hypohalite ion, or less than about 1 wt % hypohalite ion. Preferably, hypohalite ion is less than about 5 wt % or even less than about 1 wt % of the amount of halogen in the product.

Aspect 17. The method of any one of Aspects 1-16, wherein the pH of the feed is modulated in response to one or more of feed pH, product pH, a halogen concentration of the product, a current passed during electrolysis, or any combination thereof.

Aspect 18. The method of any one of Aspects 1-17, wherein (a) the pH of the feed is modulated by modulating a content of a brine acid of the halide brine, (b) the pH of the feed is modulated by modulating an amount of a feed acid of the feed, or both (a) and (b).

Aspect 19. The method of any one of Aspects 1-18, wherein the feed further comprises a buffer.

Aspect 20. The method of any one of Aspects 1-19, wherein the electrolyzing is effected by an electrolytic cell operating at an applied cell plate-to-plate voltage of from 4 to about 6 V.

Aspect 21. The method of any one of Aspects 1-20, wherein the feed water defines a pH in the range of from about 5 to about 9.

Aspect 22. The method of any one of Aspects 1-21, wherein the electrolyzing is effected in an undivided cell.

Aspect 23. A system for the production of a halogen solution, comprising: an electrolysis cell, the electrolysis cell being configured to electrolyze a feed comprising a halide brine so as to give rise to a product having a hypohalous acid concentration; a source of the halide brine, the source of the halide brine in fluid communication with the electrolysis cell; and a sensor train configured to determine any one or more of feed pH, product pH, halogen concentration of the product, a current of the electrolysis cell, a voltage of the electrolysis cell, the system being configured to modulate a pH of the feed and/or a condition of the electrolysis cell such that the hypohalous acid concentration of the product is maintained in the range of from about 1,000 to about 10,000 mg/L.

Aspect 24. The system of Aspect 23, wherein the system is configured to form the feed by contacting the halide brine with a supply of water.

Aspect 25. The system of any one of Aspects 23-24, further comprising a source of brine acid in fluid communication with the halide brine, the brine optionally being an inorganic acid.

Aspect 26. The system of any one of Aspects 23-25, further comprising a source of feed acid, the system configured to contact the feed acid and the halide brine so as to form the feed, and the feed acid optionally being an inorganic acid.

Aspect 27. The system of any one of Aspects 23-26, wherein the sensor train is configured to determine feed pH.

Aspect 28. The system of any one of Aspects 23-27, wherein the sensor train is configured to determine product pH.

Aspect 29. The system of Aspect 23, wherein the system is configured to maintain a product pH of from about 4 to about 8.

Aspect 30. The system of any one of Aspects 23-29, wherein the halide is chloride and wherein the halide is chloride and wherein the product defines a pH of from about 5 to about 6.

Aspect 31. The system of any one of Aspects 23-29, wherein the halide is bromide and wherein the product defines a pH of from about 6 to about 7.

Aspect 32. The system of any one of Aspects 23-29, wherein the hypohalous acid comprises hypochlorous acid and wherein the product defines a pH of above about 4.

Aspect 33. The system of Aspect 32, wherein the product defines a pH of from about 5 to about 6.

Aspect 34. The system of any one of Aspects 23-29, wherein the hypohalous acid comprises hypobromous acid and wherein the product defines a pH of above about 4.

Aspect 35. The method of Aspect 34, wherein the product defines a pH of from about 6 to about 7.

Aspect 36. The system of any one of Aspects 23-35, wherein the system is configured such that the product comprises an amount of halogen, and wherein the amount of halogen in the product is less than about 10 wt % (i.e., on a weight basis) molecular halogen, e.g., less than about 10 wt % molecular halogen, less than about 5% wt % molecular halogen, or less than about 1 wt % molecular halogen. Preferably, molecular halogen is less than about 5 wt % or less than about 1 wt % of the amount of halogen in the product.

Aspect 37. The method of any one of Aspects 23-36, wherein the system is configured such that the product comprises an amount of halogen, and wherein the amount of halogen in the product is less than about 10 wt % (i.e., on a weight basis) hypohalite ion, e.g., less than about 10 wt % hypohalite ion, less than about 5 wt % hypohalite ion, or less than about 1 wt % hypohalite ion. Preferably, hypohalite ion is less than about 5 wt % or even less than about 1 wt % of the amount of halogen in the product.

Aspect 38. The system of any one of Aspects 23-37, wherein the sensor train is configured to determine a halogen content of the product.

Aspect 39. The system of any one of Aspects 23-38, wherein the halide brine comprises chloride ion.

Aspect 40. The system of any one of Aspects 23-39, further comprising a source of buffer in fluid communication with the feed.

Aspect 41. The system of any one of Aspects 23-40, further comprising a tank configured to receive product of the electrolysis cell.

Aspect 42. The system of any one of Aspects 23-41, wherein the electrolysis cell is an undivided cell.

Aspect 43. A method, comprising operating a system according to any one of Aspects 23-42 so as to give rise to a product having a hypohalous acid concentration in the range of from about 1,000 to about 10,000 mg/L.

Aspect 44. The method of Aspect 43, wherein the product has a hypohalous acid concentration in the range of from about 3,000 mg/L to about 8,000 mg/L.

Aspect 45. A method of forming a halogen solution, comprising: electrolyzing an aqueous feed comprising a halogen in halide form so as to give rise to a product comprising the halogen, the halogen in the product being primarily in hypohalous acid form, as measured on a molar basis exclusive of water in the product, and the electrolyzing optionally being performed in an undivided cell; and modulating a pH of the feed and/or an operating condition of the electrolyzing such that the hypohalous acid concentration of the product is maintained in the range of from about 1,000 to about 10,000 mg/L, optionally in the range of from about 3,000 mg/L to about 8,000 mg/L.

The hypohalous acid concentration of the product can be, e.g., from about 1000 to about 10000 mg/L, or from about 1500 to about 9500 mg/L, or from about 2000 to about 9000 mg/L, or from about 2500 to about 8500 mg/L, or from about 3000 to about 8000 mg/L, or from about 3500 to about 7500 mg/L, or from about 4000 to about 7000 mg/L, or from about 4500 to about 6500 mg/L, or from about 5000 to about 6000 mg/L, and all intermediate values and subranges.

The halogen can be, e.g., one or more of bromine, chlorine, fluorine, or iodine. The halogen can be in salt form, e.g., as a sodium salt (e.g., NaBr, NaCl, NaF, NaI), as a potassium salt (e.g., KBr, KCl, KF, KI), as a lithium salt (e.g., LiBr, LiCl, LiF, LiI), as a copper salt (e.g., CuBr₂, CuCl₂, CuF₂, CuI₂), as a silver salt (e.g., AgBr, AgCl, AgF, AgI), as a calcium salt (e.g., CaBr₂, CaCl₂), CaF₂, CaI₂). The halogen can be present as multiple salts of the same halogen (e.g., as KCl and NaCl), but can also be present as different halogens (e.g., KCl and KF, NaCl and KF).

Aspect 46. The method of claim 45, wherein the aqueous feed further comprises an acid, the acid optionally comprising hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or any combination thereof.

The acid can be present in the feed water and/or in a halide brine that is combined with the feed water. The acid can also be supplied separately (i.e., not as part of the feed water and not as part of a halide brine) to the cell in which the electrolyzing is performed.

Aspect 47. The method of any one of claims 45-46, wherein the aqueous feed comprises a feed water and comprises a halide brine that includes the halogen in halide form. The feed water can be free of halogen in halide form, but this is not a requirement, as the feed water can also include halogen in halide form. Feed water can be from a municipal water supply, but can also be on-site water (e.g., water delivered from a storage tank) and in some embodiments can even comprise seawater.

Aspect 48. The method of any one of claims 45-47, wherein on a molar basis and exclusive of water, (i) less than 10% of the halogen in the product is in molecular halogen form, (ii) less than 10% of the halogen in the product is in hypohalite form, or both (i) and (ii). In some embodiments, the product is substantially free of molecular halogen and/or hypohalite halogen.

In some embodiments, less than 10% of the halogen in the product is in molecular halogen form, less than 9% of the halogen in the product is in molecular halogen form, less than 8% of the halogen in the product is in molecular halogen form, less than 7% of the halogen in the product is in molecular halogen form, less than 6% of the halogen in the product is in molecular halogen form, less than 5% of the halogen in the product is in molecular halogen form, less than 4% of the halogen in the product is in molecular halogen form, less than 3% of the halogen in the product is in molecular halogen form, less than 2% of the halogen in the product is in molecular halogen form, or even less than 1% of the halogen in the product is in molecular halogen form.

In some embodiments, less than 10% of the halogen in the product is in hypohalite form, less than 9% of the halogen in the product is in hypohalite form, less than 8% of the halogen in the product is in hypohalite form, less than 7% of the halogen in the product is in hypohalite form, less than 6% of the halogen in the product is in hypohalite form, less than 5% of the halogen in the product is in hypohalite form, less than 4% of the halogen in the product is in hypohalite form, less than 3% of the halogen in the product is in hypohalite form, less than 2% of the halogen in the product is in hypohalite form, or even less than 1% of the halogen in the product is in hypohalite form.

Aspect 49. The method of any one of claims 45-48, wherein the product has a pH of from about 4 to about 8. The product can have a pH of about 4, about 4.5, about 5, about 5.5, about 6, about 6.5, about 7, about 7.5, or even about 8. The product can have a pH of from about 4 to about 8, or from about 4.3 to about 7.7, or from about 4.5 to about 7.5, or from about 4.8 to about 7.2, or from about 5.1 to about 6.9, or from about 5.4 to about 6.6, or even from about 5.7 to about 6.3, or even about 6.

Aspect 50. The method of any one of claims 45-49, wherein the hypohalous acid comprises hypochlorous acid and wherein the product has a pH of from about 5 to about 6.

Aspect 51. The method of any one of claims 45-50, wherein the hypohalous acid comprises hypobromous acid and wherein the product has a pH of from about 6 to about 7.

Aspect 52. The method of any one of claims 45-51, wherein the pH of the feed is modulated in response to one or more of feed pH, product pH, a halogen content of the product, a current passed during electrolysis, or any combination thereof, and further optionally wherein the (a) the pH of the feed is modulated by modulating a content of a brine acid of the halide brine, (b) the pH of the feed is modulated by modulating an amount of a feed acid of the feed, or both (a) and (b). A “halogen content” can refer to a halogen concentration as well as a halogen distribution, e.g., a distribution of the different forms of a halogen in the product. For example, “halogen content” can refer to the levels of molecular halogen and hypohalite in the product.

Aspect 53. The method of any one of claims 45-52, wherein the aqueous feed further comprises a buffer, the buffer optionally comprising an inorganic buffer, the buffer further optionally comprising an inorganic phosphate-containing buffer. Such a buffer can include, e.g., hydrogen phosphate, dihydrogen phosphate, and the like.

Aspect 54. The method of any one of claims 45-53, wherein the feed water has a pH in the range of from about 5 to about 9. The pH of the feed water can be, e.g., about 5, about 5.5, about 6, about 6.5, about 7, about 7.5, about 8, about 8.5, or even about 9.

Aspect 55. The method of any one of claims 45-54, comprising maintaining the hypohalous acid concentration of the product in the range of from about 3,000 mg/L to about 8,000 mg/L. The hypohalous acid concentration can be maintained at from about 3000 to about 8000 mg/L, or at from about 3500 to about 7500 mg/L, or at from about 4000 to about 7000 mg/L, or at from about 4500 to about 6500 mg/L, or at from about 5000 to about 6000 mg/L, and all intermediate values and subranges.

Aspect 56. A system for the production of a halogen solution, comprising: an electrolysis cell, the electrolysis cell being configured to electrolyze an aqueous feed comprising a halogen in halide form so as to give rise to a product having a hypohalous acid concentration, and the electrolysis cell optionally being an undivided cell: a source of the halogen in halide form, the source of the halogen in halide form being capable of fluid communication with the electrolysis cell; and a sensor train configured to determine any one or more of feed pH, product pH, halogen content of the product, a current of the electrolysis cell, a voltage of the electrolysis cell, and the system being configured to modulate a pH of the feed and/or a condition of the electrolysis cell such that the hypohalous acid concentration of the product is maintained in the range of from about 1,000 to about 10,000 mg/L.

The hypohalous acid concentration of the product can be, e.g., from about 1000 to about 10000 mg/L, or from about 1500 to about 9500 mg/L, or from about 2000 to about 9000 mg/L, or from about 2500 to about 8500 mg/L, or from about 3000 to about 8000 mg/L, or from about 3500 to about 7500 mg/L, or from about 4000 to about 7000 mg/L, or from about 4500 to about 6500 mg/L, or from about 5000 to about 6000 mg/L, and all intermediate values and subranges.

Aspect 57. The system of claim 56, further comprising a source of acid, the system configured to contact the acid and the aqueous feed, the acid optionally being an inorganic acid. The acid can be present in the feed water and/or in a halide brine that is combined with the feed water. The acid can also be supplied separately (i.e., not as part of the feed water and not as part of a halide brine) to the cell in which the electrolyzing is performed.

Aspect 58. The system of any one of claims 56-57, wherein the sensor train is configured to determine one or more of feed pH, product pH, or a halogen content of the product.

Aspect 59. The system of claim 58, wherein the system is configured to maintain a product pH of from about 4 to about 8. The product can have a pH of about 4, about 4.5, about 5, about 5.5, about 6, about 6.5, about 7, about 7.5, or even about 8. The product can have a pH of from about 4 to about 8, or from about 4.3 to about 7.7, or from about 4.5 to about 7.5, or from about 4.8 to about 7.2, or from about 5.1 to about 6.9, or from about 5.4 to about 6.6, or even from about 5.7 to about 6.3, or even about 6.

Aspect 60. The system of any one of claims 56-59, wherein the hypohalous acid comprises hypochlorous acid and wherein the product has a pH of from about 5 to about 6.

Aspect 61. The system of any one of claims 56-60, wherein the hypohalous acid comprises hypobromous acid and wherein the product has a pH of from about 6 to about 7.

Aspect 62. The system of any one of claims 56-61, further comprising a source of buffer in fluid communication with the feed. The buffer can optionally comprise, e.g., an inorganic buffer, the buffer further optionally comprising an inorganic phosphate-containing buffer. Such a buffer can include, e.g., hydrogen phosphate, dihydrogen phosphate, and the like.

Aspect 63. A method, comprising operating a system according to any one of claims 56-62 so as to give rise to a product having a hypohalous acid concentration in the range of from about 1,000 to about 10,000 mg/L, optionally in the range of from about 3,000 mg/L to about 8,000 mg/L. The hypohalous concentration of the product can be, e.g., from about 1000 to about 10000 mg/L, or from about 1500 to about 9500 mg/L, or from about 2000 to about 9000 mg/L, or from about 2500 to about 8500 mg/L, or from about 3000 to about 8000 mg/L, or from about 3500 to about 7500 mg/L, or from about 4000 to about 7000 mg/L, or from about 4500 to about 6500 mg/L, or from about 5000 to about 6000 mg/L, and all intermediate values and subranges.

Aspect 64. The method of claim 63, wherein on a molar basis and exclusive of water, (i) less than 10% of the halogen in the product is in molecular halogen form, (ii) less than 10% of the halogen in the product is in hypohalite form, or both (i) and (ii). In some embodiments, the product is substantially free of molecular halogen and/or hypohalite halogen.

In some embodiments, less than 10% of the halogen in the product is in molecular halogen form, less than 9% of the halogen in the product is in molecular halogen form, less than 8% of the halogen in the product is in molecular halogen form, less than 7% of the halogen in the product is in molecular halogen form, less than 6% of the halogen in the product is in molecular halogen form, less than 5% of the halogen in the product is in molecular halogen form, less than 4% of the halogen in the product is in molecular halogen form, less than 3% of the halogen in the product is in molecular halogen form, less than 2% of the halogen in the product is in molecular halogen form, or even less than 1% of the halogen in the product is in molecular halogen form.

In some embodiments, less than 10% of the halogen in the product is in hypohalite form, less than 9% of the halogen in the product is in hypohalite form, less than 8% of the halogen in the product is in hypohalite form, less than 7% of the halogen in the product is in hypohalite form, less than 6% of the halogen in the product is in hypohalite form, less than 5% of the halogen in the product is in hypohalite form, less than 4% of the halogen in the product is in hypohalite form, less than 3% of the halogen in the product is in hypohalite form, less than 2% of the halogen in the product is in hypohalite form, or even less than 1% of the halogen in the product is in hypohalite form.

1. A method of forming a halogen solution, comprising:
electrolyzing an aqueous feed comprising a halogen in halide form so as to give rise to a product comprising the halogen,
the halogen in the product being primarily in hypohalous acid form, as measured on a molar basis exclusive of water in the product, and
the electrolyzing being performed in an undivided cell; and
modulating a pH of the feed and/or an operating condition of the electrolyzing such that the hypohalous acid concentration of the product is maintained in the range of from about 1,000 to about 10,000 mg/L, optionally in the range of from about 3,000 mg/L to about 8,000 mg/L.

2. The method of claim 1, wherein the aqueous feed further comprises an acid, the acid optionally comprising hydrochloric acid, sulfuric acid, phosphoric acid, sodium bisulfate, potassium bisulfate, sodium dihydrogen phosphate, disodium hydrogen phosphate, potassium dihydrogen phosphate, dipotassium hydrogen phosphate, or any combination thereof.

3. The method of claim 1, wherein the aqueous feed comprises a feed water and comprises a halide brine that includes the halogen in halide form.

4. The method of claim 1, wherein on a molar basis and exclusive of water, (i) less than 10% of the halogen in the product is in molecular halogen form, (ii) less than 10% of the halogen in the product is in hypohalite form, or both (i) and (ii).

5. The method of claim 1, wherein the product has a pH of from about 4 to about 8.

6. The method of claim 1, wherein the hypohalous acid comprises hypochlorous acid and wherein the product has a pH of from about 5 to about 6.

7. The method of claim 1, wherein the hypohalous acid comprises hypobromous acid and wherein the product has a pH of from about 6 to about 7.

8. The method of claim 1, wherein the pH of the feed is modulated in response to one or more of feed pH, product pH, a halogen content of the product, a current passed during electrolysis, or any combination thereof, and further optionally wherein the (a) the pH of the feed is modulated by modulating a content of a brine acid of the halide brine, (b) the pH of the feed is modulated by modulating an amount of a feed acid of the feed, or both (a) and (b).

9. The method of claim 1, wherein the aqueous feed further comprises a buffer, the buffer optionally comprising an inorganic buffer, the buffer further optionally comprising an inorganic phosphate-containing buffer.

10. The method of claim 1, wherein the feed water has a pH in the range of from about 5 to about 9.

11. The method of claim 1, comprising maintaining the hypohalous acid concentration of the product in the range of from about 3,000 mg/L to about 8,000 mg/L.

12. A system for the production of a halogen solution, comprising:
an electrolysis cell,
the electrolysis cell being configured to electrolyze an aqueous feed comprising a halogen in halide form so as to give rise to a product having a hypohalous acid concentration, and
the electrolysis cell being an undivided cell;
a source of the halogen in halide form,
the source of the halogen in halide form being capable of fluid communication with the electrolysis cell; and
a sensor train configured to determine any one or more of feed pH, product pH, halogen content of the product, a current of the electrolysis cell, a voltage of the electrolysis cell, and
the system being configured to modulate a pH of the feed and/or a condition of the electrolysis cell such that the hypohalous acid concentration of the product is maintained in the range of from about 1,000 to about 10,000 mg/L.

13. The system of claim 12, further comprising a source of acid, the system configured to contact the acid and the aqueous feed, the acid optionally being an inorganic acid.

14. The system of claim 12, wherein the sensor train is configured to determine one or more of feed pH, product pH, or a halogen content of the product.

15. The system of claim 14, wherein the system is configured to maintain a product pH of from about 4 to about 8.

16. The system of claim 12, wherein the hypohalous acid comprises hypochlorous acid and wherein the product has a pH of from about 5 to about 6.

17. The system of claim 12, wherein the hypohalous acid comprises hypobromous acid and wherein the product has a pH of from about 6 to about 7.

18. The system of claim 12, further comprising a source of buffer in fluid communication with the feed.

19. A method, comprising operating a system according to claim 12 so as to give rise to a product having a hypohalous acid concentration in the range of from about 1,000 to about 10,000 mg/L, optionally in the range of from about 3,000 mg/L to about 8,000 mg/L.

20. The method of claim 19, wherein on a molar basis and exclusive of water, (i) less than 10% of the halogen in the product is in molecular halogen form, (ii) less than 10% of the halogen in the product is in hypohalite form, or both (i) and (ii) essentially free of at least one of molecular halogen and hypohalite.
